(19) United States
(12) Patent Application Publication
(10) Pub. No.: US 2019/0314682 A1
(43) Pub. Date: Oct. 17, 2019

(54) METHOD AND SYSTEM FOR AUTOMATICALLY SELECTING PHYSICAL EXERCISES

(71) Applicant: MYOTEST SA, Sion (CH)

(72) Inventors: Patrick FLACTION, Chandolin-près-Savièse (CH); Cyrille GINDRE, Aigle (CH)

(21) Appl. No.: 16/453,632

(22) Filed: Jun. 26, 2019

Related U.S. Application Data
(63) Continuation of application No. 14/831,474, filed on Aug. 20, 2015.

(30) Foreign Application Priority Data
Aug. 21, 2014 (CH) CH01257/14

(51) Int. Cl. A63B 24/00; G09B 5/00; A61B 5/11

(52) U.S. Cl. CPC A63B 24/0075; G09B 5/00; A61B 5/6838; A61B 5/1118; A61B 2505/09; A63B 24/0062

(57) ABSTRACT

The present invention relates to a method for automatically selecting exercises to propose to a user (7) carrying a mobile device (5) comprising a processor (23), wherein the user (7) also wears a motion sensor, for example an accelerometer (3) configured for communicating with the mobile device (5).

The method comprises the following steps:
classifying a predefined collection of exercises in a database;
automatic selection by the mobile device (5) and/or by the accelerometer (3),
   wherein this selection takes into account at least one feedback from the user.

The feedback indicates the sensations of the user (7) whilst performing an exercise.

### REFERENCE DATA

The present invention claims the priority of Swiss Patent Application CH20140001257, filed on Aug. 21, 2014, the content of which is incorporated here by reference.

### TECHNICAL FIELD

The present invention relates to a method and a system for automatically selecting physical exercises to be proposed to a user, for example an athlete, an elderly person or any other person.

### STATE OF THE ART

An athlete wishing to improve athletic performance, a person wishing to become slimmer or to improve wellness, or an elderly person wishing to climb stairs more efficiently are non-limiting examples of people having to execute physical exercises.

In the context of this invention, the term “physical exercise” designates training exercises, rehabilitation exercises or in general all physical exercises corresponding to any situation of at least one part of a person's body being set into movement. These exercises are generally proposed to a person for sports, rehabilitation or health purposes.

Physical exercises are often proposed by a coach or expert, who decides not only the training or rehabilitation plan for the person to be trained or rehabilitated (athlete, elderly person etc.) but also determines with this person the aim of this plan, e.g. to enhance responsiveness, to lose weight, to improve legs' coordination etc.

In other words, the coach imposes the exercises to a person to be trained along a “top-down” model.

US2007219059 describes a method and a device for monitoring and proposing an exercise plan that is continuously updated. A user can obtain a training plan by means of an exercise database, serving as exercise catalogue to a coach who can remotely propose a personalized training plan. In this case too, it is the coach who decides the personalized training plan for the user.

Document EP2263534, filed by the applicant, describes a method for optimizing training plans, wherein accelerations of the user are measured during an initial test by a mobile device in order to calculate parameters representing the muscular characteristics of the user. Exercises adapted to these muscle characteristics are determined and proposed to the user. These exercises can be selected automatically in order to improve the weakest muscle parameters.

However, this selection aims only to improve the user's weakest muscle parameters, without taking into account the user's objectives. For example, a sprinter could be content with limited endurance and will need to improve explosiveness and muscular power even if these physical attributes are already very good. Conversely, a user whose sole objective is to lose weight would perhaps not increase muscle mass and power even if these parameters are insufficient.

A good coach usually takes into account the user's feeling during the execution of the exercises; the coach thus does not rely only on the physical assessment of the user. Conversely, the automatic systems for recommending exercises as known in the prior art do not take this feeling into account. Even if the exercises proposed are theoretically adapted for improving a weak muscular parameter of the user, there is no guarantee that this user who in practice performs these exercises will be comfortable whilst performing a physical exercise. The user has thus no means to indicate whether they feel good during the execution of a physical exercise.

In the context of this invention, the expression “sensation of the user during the performance of an exercise” indicates the feelings, emotions and in generally what the user perceives during the execution of this exercise. For example, a user can be comfortable, i.e. feel good, when running in an asymmetric manner, for example by using the right leg more than the left leg, even if this asymmetric running style will probably never be advised by a coach or an automatic training exercise selection system. A user can also feel well when running first slowly and then accelerating at the end, rather than starting running fast before slowing down. Finally, some users feel comfortable by repeating the same exercise several times whilst others prefer to change frequently the type of exercise performed.

GB2447915 describes a device comprising a database for creating a personalized training plan corresponding to the sport and characteristics of the user. The device comprises a multimedia device and a database that, based only on the answers to an application questionnaire, enable the user or the coach to create a training plan. The exercises are thus proposed without taking into consideration the muscle parameters measured.

### BRIEF SUMMARY OF THE INVENTION

One aim of the present invention is to propose a method and a system for automatically selecting more personalized physical exercises and that take into account the objectives, the level and the sensations of the users during the performance of the physical exercises.

Another aim is to propose a method and a system for automatically selecting physical exercises that allows the user who performs these exercises to feel comfortable and/or to progress.

Another aim of the present invention is to propose a method and a system for automatically selecting physical exercises that are not based on the “top-down” model discussed.

Another aim of the present invention is to propose a method and a system for automatically selecting exercises improved as compared with the known methods and systems.

According to the invention, these aims are achieved notably by means of a method for automatically selecting physical exercises to propose to a user, wherein said user wears a motion sensor, for example an accelerometer, a gyroscope or other, wherein the method comprises the following steps:
classifying a predefined collection of physical exercises in a database according simultaneously to the user's objectives, to the physical attributes that these physical exercises allow to reinforce, and for each physical attribute the levels for which these physical exercises are appropriate;
inputting by the user of personal data comprising at least one user objective;
testing by means of said motion sensor of the user's level for different physical attributes;
automatically selecting at least one physical exercise in said database on the basis of said user objective entered by the user, of one or several physical attributes to be reinforced and/or activated as determined according to the user's level for this attribute, and at least one user feedback, said feedback indicating the user's sensation during the performance of a physical exercise.

In the context of this invention, the expression “user objective” refers to the user's purpose for executing physical exercises. A user objective can be very general (for example “to lose weight”) or else correspond to a particular sport (for example “train for the marathon”).

The user objectives can thus include a sport for which the user wishes to train.

The user objectives can also include user preferences (for example “I don't like doing repetitive exercises”) and/or the physical attributes the user prefers or likes to improve.

In one variant embodiment, the feedback includes a haptic or vocal command deliberately entered by the user during the performance of a physical exercise in order to indicate a sensation.

This solution makes it possible notably to propose more personalized physical exercises taking into account notably the user objectives articulated by the user when inputting the initial personal data.

This solution also has the advantage over the prior art of making use of the user's feedback, on the basis of a “bottom-up” model: the system according to the invention in fact takes into account the reaction, not only physical but also emotional, of the user performing the exercises and which is used for choosing the exercises to be suggested. In other words, instead of the exercise selection being imposed from the top (an electronic coach), it takes into account the aspirations from the bottom (the user) in consideration of the user's sensation whilst performing these exercises.

The system according to the invention thus makes it possible to arrive at the training or rehabilitation objective chosen by the user thanks to a selection of exercises taking into account the user's experience, even if these exercises are not the most efficient to achieve this objective from a theoretical point of view.

In one embodiment, the feedback includes a signature of the user's movement determined automatically on the basis of acceleration data collected by the accelerometer. In other words, the automatic exercise selection can be achieved depending on the signature of the movement of the user during the execution of a physical exercise.

The signature of the movement of the user comprises the temporal evolution of at least one of the following parameters and/or a combination of at least two of the following parameters: strength, power, speed, stability, reaction time, flight time, contact time, stiffness, responsiveness, asymmetry, inclination, fatigue, risk of injury, (motor and/or energetic) gesture efficiency. These parameters can be simple, i.e. measured directly (for example the contact time), or complex, because they are obtained from the integration or in general from the computations of the simple parameters (for example fatigue). In another embodiment, the movement signature is read, i.e. it is compared to a time curve expected depending on the performed exercise.

The physical attributes comprise at least two of the following physical attributes: “core stability”, endurance, flexibility, power, agility, perception. The “core stability” attribute comprises strengthening core muscles for stability and balance. The physical attribute “power” can include power of the user's lower body (e.g. the legs) and/or power of the upper body (for example arms and/or trunk). The physical attribute “power” can include force and speed. The physical attribute “agility” can include balance, reaction and/or rhythm. In another variant embodiment, the physical attributes also comprise perception, i.e. the user's reaction to any type of signal, for example an audio and/or visual signal and/or visual discrimination ability. In one embodiment, the physical attribute “agility” also comprises perception.

The levels are at least two and are indicative, in a progressive fashion, of the user's physical attributes and/or of the difficulty of each physical exercise.

In a variant embodiment, the method according to the invention comprises the following steps:
measuring the user's level for at least one physical parameter during the performance of the selected physical exercises by the system according to the invention, and
subsequent selection of new physical exercises according to this level, the user objectives and the user's feeling. In one variant embodiment, this later selection also depends on the signature of the movement of the user.

In one variant embodiment, the personal data comprise at least one of the following details: age, weight, height, gender of the user. In another embodiment, these data also comprise a pathology, i.e. a possible illness and/or physical problem of the user and/or the user's state of recovery, which is useful notably in a reeducation context during and/or after an illness of the user.

Advantageously, the initial test proposed to the user can evolve and change automatically depending on the different physical attributes measured during the performance of the physical exercises.

In a preferred embodiment, one or several physical exercises can constitute a training unit, which has a duration of several minutes, for example 10 minutes. In the context of this invention, the word “session” then indicates a training unit, or an assembly of several training units. A session can thus have a total duration of a whole number of weeks, for example 3 weeks. In one variant embodiment, reference exercises are integrated in the sessions in order to cause the training to evolve according to the level achieved at the time t+1 as compared to the level reached at the time t. In another variant, each session is followed by an assessment test.

According to the invention, these aims are achieved also by means of a system for automatically selecting physical exercises to propose to a user, comprising:
a motion sensor designed for being worn by the user,
a database in which a predefined collection of physical exercises is classified according simultaneously to the user's objectives, to the physical attributes that these physical exercises allow to reinforce and/or activate, and for each physical attribute to the user levels for which these physical exercises are appropriate,
wherein the system is configured to allow the user to input personal data comprising at least one user objective and to automatically select at least one physical exercise in the database on the basis of the user objective entered by the user, of one or several physical attributes to be reinforced and/or activated as determined according to the user's level for this attribute, and at least one user feedback, said feedback indicating the user's sensation during the performance of a physical exercise.

In a preferred embodiment, the user also carries a mobile device.

In a preferred embodiment, the motion sensor is worn close to the user's center of mass. In a preferred embodiment, the motion sensor is an accelerometer, for example a three-axis accelerometer. In another embodiment, as alternative or complement to the previous one, the motion sensor is a gyroscope.

In another embodiment, the relation between the mobile device and the accelerometer is of the master-slave type.

### BRIEF DESCRIPTION OF THE FIGURES

Examples of embodiments of the invention are indicated in the description illustrated by the attached figures in which:

FIG. 1 illustrates a user carrying or wearing a mobile device and an accelerometer of the automatic exercise selection system according to one embodiment of the invention.

FIG. 2 illustrates an embodiment of the mobile device and of the accelerometer of the automatic exercise selection system of FIG. 1.

FIG. 3A illustrates an embodiment of the levels of the automatic exercise selection method of the invention.

FIG. 3B illustrates an embodiment of the two-dimensional grid (levels—physical attributes) of the automatic exercise selection method of the invention.

FIG. 3C illustrates an embodiment of a space with three dimensions (levels—physical attributes—exercises) of the automatic exercise selection method of the invention.

FIG. 4 illustrates an embodiment of correcting the levels of each physical attribute after the initial test of the automatic exercise selection method of the invention.

### EXAMPLE(S) OF EMBODIMENTS OF THE INVENTION

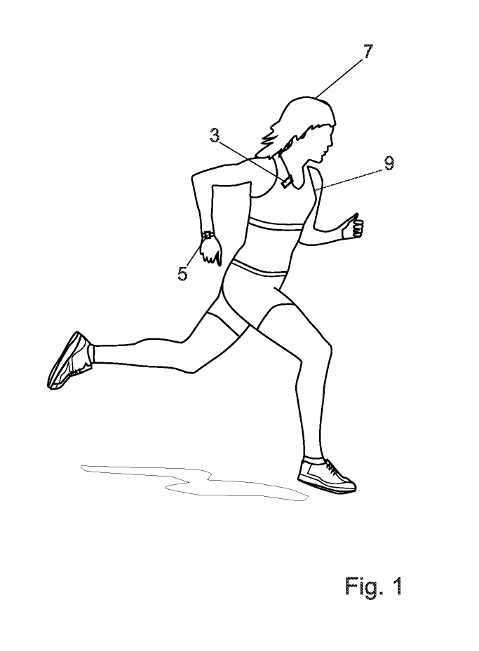

The present invention relates to a method for automatically selecting physical exercises to be proposed to a user. As illustrated in FIG. 1, this user 7 carries or wears a mobile device 5, in the illustrated example a watch, and a motion sensor, for example an accelerometer 3 configured for communicating with the mobile device 5. In the following description given by way of example, for the sake of simplicity, reference will be made to an accelerometer as motion sensor. However, it must be understood that the invention is not limited to such a sensor but also includes other motion sensors, for example a gyroscope etc. In a preferred embodiment, the communication between the mobile device 5 and the accelerometer 3 is wireless.

The mobile device 5 is not necessarily a watch but can be, for example an in a non-limiting way, a portable music reader, a smartphone, spectacles, an item of jewelry etc. or any device that can be carried by a user. The mobile device 5 and the accelerometer 3 can also be integrated within a single object.

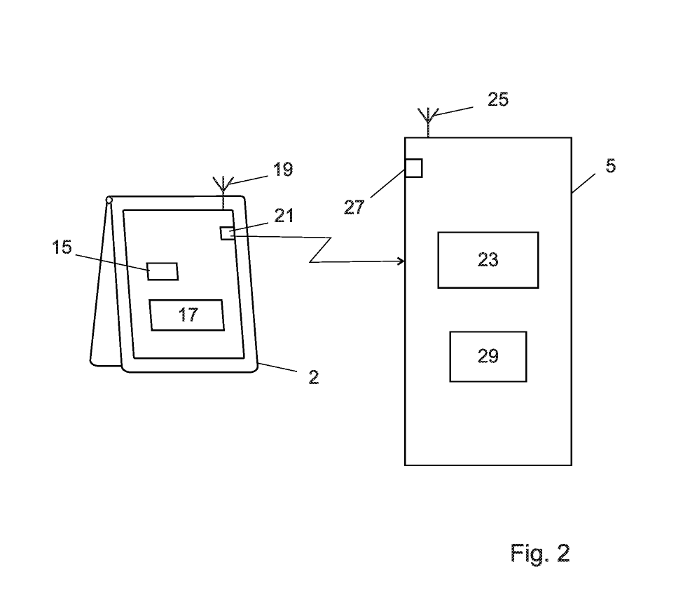

Advantageously, the mobile device 5 comprises a processor 23, visible in FIG. 2, configured for compiling acceleration data that are transmitted to it from the accelerometer 3, a memory 29 configured for storing data or the results of the compiling of the processor 23, a receiver 25 and a transmitter 27 to communicate with the accelerometer.

The accelerometer 3 is configured for being connected in a removable fashion, for example using clips 2, a ventral belt, a bracelet etc. to the clothing 9 of the user 7 or directly to the user 7. In another embodiment, the accelerometer is in one shoe or connected to one shoe of the user 7.

Advantageously, the accelerometer 3 comprises means for measuring the acceleration 15 (acceleration sensor, for example tri-axial), visible in FIG. 2, a memory 15, a receiver 19 and a transmitter 21 for communicating with the mobile device 5. The accelerometer can also comprise a processor not represented.

Advantageously, the mobile device 5 serves as master relative to the accelerometer 3 (slave), since it can request acceleration data to the accelerometer 3 only during certain events, such as described in document WO2012171967 filed by the applicant and the contents of which are incorporated hereto by reference.

The method according to the invention comprises the following steps:
classifying a predefined collection of exercises 300 in a database according simultaneously to user objectives previously selected by the user 7, to the physical attributes 200 that each exercise 300 allows to reinforce and/or activate, and to levels 100 of the user 7 and/or of the exercise 300 for each physical attribute 200;
automatic selection by the mobile device 5 and/or by the accelerometer 3 of at least one exercise in the database.

In another embodiment, the selection is achieved by any other computation means such as a PC, a laptop, a tablet etc. that is not carried or worn by the user and which is connected to the mobile device 5 and/or to the accelerometer 3 via a wired or wireless connection.

This automatic selection is advantageously effected on the basis of the user objective entered by the user 7, of one or several physical attributes 200 to be reinforced and/or activated determined according to the level 100 of the user for this attribute as measured by means of the accelerometer 3, and of at least one user feedback.

The user objectives selected by the user can be chosen from a list of predefined user objectives. As discussed, these can be general objectives, such as “get fit”, “weight loss”, or objectives linked to a sport and/or a reeducation pathology, for example “ski training”, “preparing for a marathon”, “training to rehabilitate the left knee” etc.

Advantageously, the feedback from the user 7 indicates the sensation of the user 7 whilst performing an exercise. As discussed, in the context of this invention, the expression “sensation of the user during the performance of an exercise” indicates the “mood”, the feelings, emotions and in generally what the user 7 perceives during the execution of this exercise.

Advantageously, the classification of a predefined collection of exercises in a database is carried out beforehand by a coach or an expert on the basis of his knowledge and know-how.

The exercises 300 can be classified according to:

1. the levels 100 that the exercise requires for each physical attribute;

2. the physical attributes 200 that these exercises allow to reinforce and/or activate;

3. the user objectives selected by the user 7.

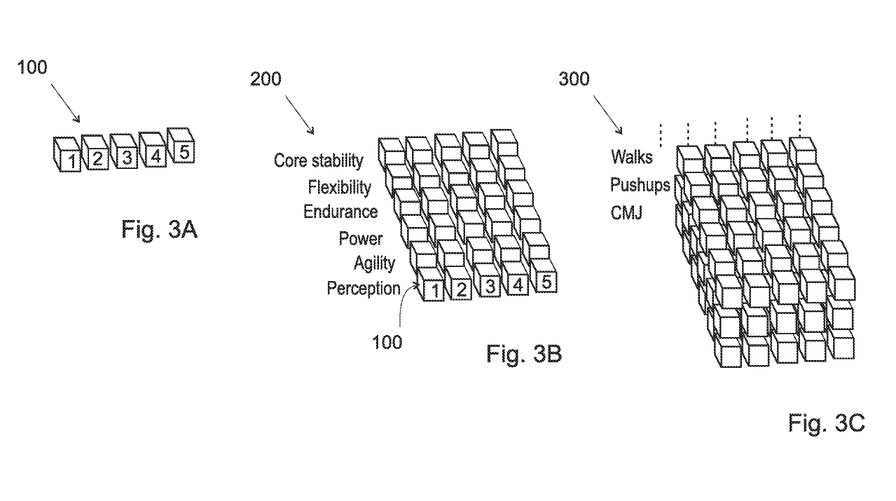

The number of levels 100 is a whole number, for example 5 as illustrated in FIG. 3A, wherein level 1 is for example the easiest level, level 5 is for example the hardest level, and intermediate levels 2 to 4 represent in a progressive manner an increase in difficulty.

These five levels are used for classifying the different users according to the results they achieve for each physical attribute during an initial test. Thus, a person starting out with running will reach level 1 for the physical attribute “endurance”; an elite marathon-runner will achieve level 5.

The different exercises 300 stored in the database can also be associated to levels 100. Thus a 400 m lap at a light pace will be adapted to users at level 1 or 2 of the physical attribute “endurance”; a quick-paced training over 20 kilometers is suitable for users at level 4 or 5 of this physical attribute.

A same exercise 300 can be associated to different physical attributes 200 and different levels 100. For example, an uphill run will be useful for improving both the endurance and the power of the lower limbs.

In a preferred embodiment, it is possible to switch to a level that precedes or follows it by increments of 1 (for example, it is possible to move from the level 2 to the level 1 or 3, but not directly to level 4). In another preferred embodiment, the increments can be smaller, for example 0.1, which gives a greater resolution for classifying the exercises. In another embodiment, it is possible to jump from one level to any other level.

In a preferred embodiment, there are six physical attributes 200 that the exercises allow to reinforce and/or activate, namely:

1. “core stability”, which comprises the notion of strengthening core muscles for stability and balance,
2. flexibility,
3. endurance,
4. power, which comprises lower power (i.e. of the user's lower body, e.g. the legs) and/or upper power (i.e. of the user's upper body, for example arms),
5. agility, which includes for example coordination,
6. perception, which comprises any physical reaction to a signal.

By using the levels 100 and the physical attributes 200, it is thus possible to classify the exercises in a two-dimensional space (level x physical attribute), of which one embodiment is illustrated in FIG. 3B. For each exercise, this classification makes it possible to allocate at least one level 100 for each physical attribute 200 that the exercise allows to train.

This two-dimensional space can advantageously be represented in the form of a grid or table, having on the horizontal axis the levels 100 and on the vertical axis the physical attributes 200.

Each exercise is classified in this grid by specifying the percentage of each physical attribute that is required for a given level. The sum of the percentages of the grid must be 100%.

By way of example, hereafter is the two-dimensional grid for the counter-movement jump (CMJ) with free arms:

This grid indicates that the free-arm CMJ exercise enables the physical attributes “lower power” and “upper power” to be exercised. This grid shows notably that this exercise is appropriate for a user who has an average level (level 3) for the physical attribute “lower power” and a level slightly lower than average (level 2) for the “upper power”. The percentages indicate that this exercise contributes for 90% to improving and/or loading lower power and for 10% to improving the upper power.

A same exercise can be recommended to different types of users having different levels for one or several physical attributes. For example, the exercise here above can also be adapted to a user having a level 2 for “lower power” and level 1 for the “upper power”.

This classification of each exercise in this two-dimensional grid corresponds to the adjunction of a third dimension, as is visible in FIG. 3C. In fact, each exercise 300 (walking, pushups, CMJ etc.) corresponds to a two-dimensional grid as illustrated here above.

The marking or classification of each exercise 300 in the two-dimensional space of FIG. 3B also depends on:
the type of exercise;
of an instruction given with the exercise, for example the number of times the exercise is to be repeated, the load that needs to be displaced, the duration of the rest periods between each repetition etc. For example, an exercise of the type CMJ can be classified differently depending on whether the instruction is to repeat it once or several times or to perform it with an additional load on the body.

In a preferred embodiment, each user objective can also be classified in a two-dimensional grid such as that represented in FIG. 3B.

For example, the two-dimensional grid hereafter for a user objective “anti-ageing”:

This grid indicates that this objective contributes for 20% to improving agility, for 20% to enhancing endurance, for 10% to developing flexibility etc. This grid shows that in order to achieve the objective, it is necessary to dedicate 20% of the training units to agility, 20% to endurance, 10% to flexibility, etc.

The following rules can be used to modify this classification:
the more the age of the user 7 increases, the more the required levels of physical attributes diminish;
in the case of a female person, the level of required core muscles, power (lower and upper) and endurance is reduced;
if the BMI is higher than the average BMI of a person of the same gender and age as the user 7, the theoretical levels of the physical attributes diminish.

The method according to the invention advantageously provides rules enabling the user 7, notably his/her characteristics, the exercises 300 and the user objectives to be connected.

Prior to starting to perform exercises, the user enters personal data into the mobile device 5, in the accelerometer 3 or in any other device connected, in a wired or wireless fashion, with the mobile device 5 or the accelerometer 3, for example a computer, PDA, laptop etc.

In a preferred embodiment, these data comprise the name of the user 7 as well as:
the age
the gender
the weight and height (necessary for calculating the BMI—Body Mass Index)
the initial objective (for example getting fit, wellness, sports training etc.). The inputted objective can indicate the training for a particular sport chosen from among a list of sports.
   In one embodiment, these data also comprise a pathology, i.e. a possibly illness and/or physical problem and/or state of recovery of the user, which is useful notably in a context of reeducation of the user during and/or after an illness.

The system then proposes n or several initial exercises to the user.

Whilst performing these initial exercises, the accelerometer 3 measures accelerations that are transmitted to the mobile device 5. The tests proposed (reference exercises) can depend on the personal data entered by the user. For example, an elderly and corpulent user will not get the same initial tests (reference exercises) for measuring endurance offered as a young user with the aim of training for running.

In a preferred embodiment, these initial exercises are selected on the basis of the user's age, gender, BMI, pathology and initial objective.

The initial physical tests are preferably constituted by a series of rather short exercises, comprising for example a series of jumps, a short run etc.

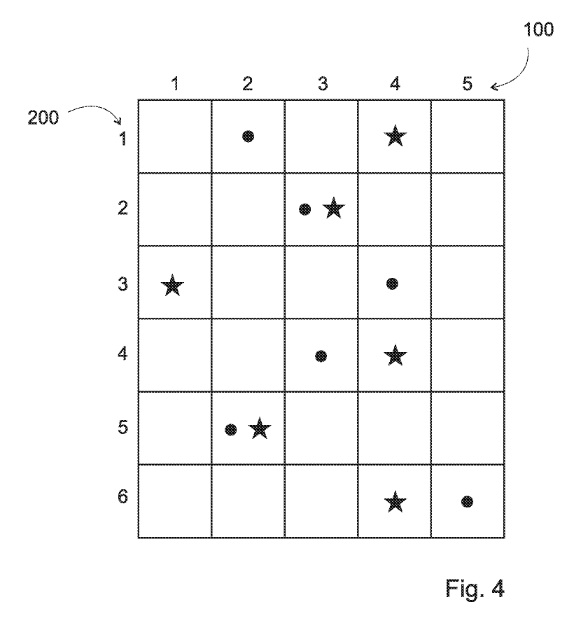

The personal data entered by the user 7 make it possible to determine the theoretical level of the user 7 for each predetermined physical attribute. This classification can be done by the mobile device 5 (or by a device connected to the mobile device 5) and allows, for each physical attribute, a theoretical level to be determined going for example from 1 (beginner) to 5 (expert). In other words, each user 7 is classified in a two-dimensional grid similar to that of FIG. 3B which was used for classifying exercises and which is now used for classifying the user 7. This theoretical level is represented by round dots in FIG. 4.

Measurements carried out during the initial physical tests, for their part, allow the physical attributes of the user 7 (represented by stars in FIG. 4) to be calculated and the measured physical attributes to be compared to those calculated theoretically on the basis of the data entered by the user 7 (represented by circles in FIG. 4).

Thanks to this comparison, it is possible to direct the stimulation of the physical attributes 200 either by being oriented on the user's weaknesses (with the aim of reducing the gap between the circles and the stars in FIG. 4) or by preferably using the user's forces (with the aim of focusing the preparation on the attributes for which the stars are of a higher level than the circles). In other words, the first association of physical attributes 200 to levels 100 based only on the data inputted by the user 7 is not static but dynamic and takes into account the levels of the physical attributes measured during the performance of the initial physical tests (or reference tests).

In another embodiment, it is not only the theoretical levels of each physical attribute that are modified, but the type of initial exercise proposed to the user can also be modified.

The system is thus evolving: in other words, the a priori pinpointing of the level of each physical attribute of the user (the circles) is not fixed but changes over time since the system according to the invention evolves depending on the levels measured for the previous users and/or for the same user.

The initial test also evolves over time, according to the parameters measured by previous users during the initial test.

In a preferred embodiment, the user, when inputting the personal data, can also indicate the physical attribute 200 s/he prefers or wishes to improve: in this case, the percentage (or relative weighting) of this physical attribute will be increased accordingly.

Once the user 7 has performed the initial exercises, exercises are automatically selected in the space illustrated in FIG. 3C depending on the objectives selected by the user 7 (for example the sport for which s/he is training or the body part that is to be reeducated) and one or several physical attributes 200 to be reinforced and/or activated, determined according to the level 100 of the user 7 for this attribute.

Advantageously, the mobile device 5 comprises means for inputting a haptic or vocal command during the performance of a physical exercise, wherein this command indicates the sensation of the user 7 during this exercise. In other words, this command constitutes a feedback that is also taken into account in the inventive system for selecting the exercises proposed to the user 7.

In another embodiment, the accelerometer 3 and not the mobile device 5 comprise these means for inputting a haptic and/or vocal command of the user 7.

In the context of this invention, the expression “haptic command means” indicates any element or means that performs a function if it is touched by a user or by a means such as a stylus. Examples of haptic control element include pushbuttons or a touch screen. The means for inputting a voice command can include a microphone.

In a preferred embodiment, these means comprise two haptic command means, for example two buttons, one button for entering a command corresponding to a positive sensation (“I like”, “I feel good”) and one button for entering a negative sensation (“I don't like”).

In another variant embodiment, there is only a single button for entering a command corresponding to a positive sensation.

In another embodiment, the number of haptic command means is greater than two, in order to consider intermediate nuances between the sensations of “I like” and “I don't like”.

In another variant, the mobile device 5 and/or the accelerometer 3 comprise means for inserting a voice command from the user: expressions such as “I like it” etc. can thus be recognized and used by the inventive system as feedback from the user 7 to automatically select exercises. In another embodiment, the command is detected by the accelerometer 3 on the basis of impulsions detected by it: for example, the user can directly tap on the accelerometer 3 a whole number of times, for example twice, to indicate s/he likes the exercise that is being performed. In a preferred embodiment, two quick taps, with the two impulsions being separated by a time range of less than 1 second, for example less than 0.5 seconds, are used to indicate the exercise is liked.

Using the user's feedback makes it possible to take into account the reaction, not only physical but also emotional, of the user 7 performing exercises, which is used to select the exercises to be proposed to that user.

For example, the system according to the invention can measure that during a run, the user uses the legs in an asymmetrical fashion: one exercise can thus be proposed to try and correct this asymmetry. If, however, the user likes to run in this asymmetric manner, it is possible for him/her to enter the command “I like” to the system that thus no longer tries to correct the asymmetry, since the user feels comfortable when running in this way.

In other words, the system according to the invention makes it possible to achieve the objective of training or activation or pleasure or also rehabilitation by taking into account the manner in which the user 7 feels comfortable, even if this manner is not the most efficient from a theoretical point of view.

In another variant, the mobile device 5 and/or the accelerometer 3 enable the signature of the movement of the user to be determined on the basis of acceleration data collected by the accelerometer 3.

In the framework of this invention, the expression “signature of the movement” indicates how the measured acceleration values vary over time. It also indicates how parameters calculated on the basis of accelerations, or a combination of these parameters, vary over time. Examples of these parameters, simple or complex, are: force, power, speed, stability, reaction time, flight time, contact time, stiffness, responsiveness, asymmetry, inclination, fatigue, risk of injury.

Thus, the system according to the invention will propose different exercises to two users 7 having the same objectives but different movement signatures.

Advantageously, the database of exercises proposed to the user thus evolves, i.e. it is not static but changes over time.

During the execution of these exercises, the accelerometer 3 measures accelerations that are transmitted to the mobile device 5, whose processor 23 is designed for calculating muscle parameters on the basis of acceleration data. In another variant, the accelerometer 3 comprises a processor for directly calculating these muscle parameters.

In a preferred embodiment, these muscle parameters comprise:
force
power
speed
stability
reaction time
flight time and duration of contact with the ground
stiffness
responsiveness
asymmetry
repeatability
number of repetitions
inclinations/angles
reaction
rhythm
height
balance
oscillation
any combination of two or several of the above parameters.

Documents EP2582295 and EP2582294, filed by the applicant and also incorporated by reference, describe examples of muscle parameters that can be calculated by the system according to the invention.

According to an independent aspect of the invention, the inventive system allows the mobile device 5 and/or the accelerometer 3 to automatically determine the user's objectives. In other words, in the known systems, the user 7, on his own and/or with the aid of a coach, would determine the training and/or rehabilitation objectives. In a variant embodiment of the present invention, the mobile device 5 and/or the accelerometer 3 are able to determine this objective, on the basis of:
the personal data entered by the user 7 (age, gender, etc.);
the measurements made by the accelerometer;
the selected user objective;
the user's feedbacks (sensations during the performance of the exercise and/or the signature of the movement).

In a preferred embodiment, the automatic determination of the objectives also evolves whilst the user 7 executes the exercises.

In a preferred embodiment, the exercises proposed to the user 7 constitute a training unit, which has a duration of several minutes, for example 10 minutes. In the context of this invention, the word “session” then indicates a training unit, or an assembly of several training units. A session can thus have a total duration of a whole number of weeks, for example 3 weeks. In one variant embodiment, reference exercises are integrated in the sessions in order to cause the training to evolve according to the level achieved at the time t+1 as compared to the level reached at the time t. In another variant, each session is followed by an assessment test.

In one variant, the sessions are automatically composed so as to comply with the following rules:
if the test results are lower than expected for a given physical attribute, exercises respecting the test results are proposed to the user 7, for example exercises from a lower level;
the level of difficulty of the selected exercises will strive to not exceed by one point the tested level;
the relative weighting of each physical attribute stimulated by all of the exercises of a session complies with the user objective marker and/or the user objective and/or the weighting parameters (sensations, movement signature . . . ).

To sum up, it is thus possible to classify and select exercises on the basis of the level of the exercise and/or of the user, of the physical attributes to improve, of the selected user objective, of the user's sensation during the performance of the exercise but also on the basis of the signature of the movement of the user.

1. Method for automatically selecting physical exercises to propose to a user, wherein said user wears a motion sensor, for example an accelerometer,
wherein the method comprises the following steps:
classifying a predefined collection of physical exercises in a database according simultaneously to the user's objectives, to the physical attributes that these physical exercises allow to reinforce and/or activate, and for each physical attribute the levels for which these physical exercises are appropriate;
inputting by the user of personal data comprising at least one user objective;
testing by means of said motion sensor of the level of the user for different physical attributes;
automatically selecting at least one physical exercise in said database on the basis of said user objective entered by the user, of one or several physical attributes to be reinforced and/or activated as determined according to the level of the user for this attribute, and of at least one user feedback, said feedback indicating the sensation of the user during the performance of a physical exercise.

2. The method according to claim 1, wherein said feedback includes a haptic or vocal command deliberately entered by the user during the performance of a physical exercise in order to indicate a sensation.

3. The method according to claim 1, wherein said feedback includes a signature of the movement of the user determined automatically on the basis of acceleration data collected by said accelerometer.

4. The method according to claim 1, wherein the physical attributes comprise at least two of the following physical attributes: core stability, flexibility, endurance, power, agility, perception.

5. The method according to claim 1, wherein the levels are at least two and indicate in a progressive manner the physical attributes of said user and/or the difficulty of each physical exercise.

6. The method according to claim 1, wherein said user objectives include a sport that the user wishes to train and/or a pathology.

7. The method according to one claim 3, wherein said automatic selection is effected on the basis of said signature of the movement of said user during the performance of a physical exercise.

8. The method according to one claim 1, comprising the measuring of the level of the user for at least one physical parameter and/or a pathology during the performance of the selected physical exercises, and a subsequent selection of new physical exercises according to this level, of the user objectives and of the user's feeling.

9. The method according to claim 3, wherein said signature of the movement of said user comprises the evolution over time of at least one of the following parameters and/or a combination of at least two of the following parameters: force, power, speed, stability, reaction time, flight time, contact time, stiffness, responsiveness, asymmetry, inclination, fatigue, risk of injury, number of repetitions, reaction, rhythm, height, balance, oscillation.

10. The method according to claim 1, wherein said personal data comprise at least one of the following data: age, weight, height, gender of the user, pathology.

11. The method according to claim 1, comprising the evolution and automatic modification of said test according to the different physical attributes measured during the execution of the physical exercises.

12. The method according to claim 1, wherein one or several physical exercises are regrouped in a training unit, wherein one or several training units are regrouped in sessions having a total duration of a whole number of weeks, for example 3 weeks, wherein reference exercises are integrated in each session so as to cause the training to evolve according to the level achieved at the time t+1 as compared to the level reached at the time t.

13. System for automatically selecting physical exercises to propose to a user, comprising:
a motion sensor, for example an accelerometer designed for being worn by said user,
a database in which a predefined collection of physical exercises is classified according simultaneously to the user's objectives, to the physical attributes that these physical exercises allow to reinforce and/or activate, and for each physical attribute to the levels of users for which these physical exercises are appropriate,
wherein the system is configured to allow the user to input personal data comprising at least one user objective and to automatically select at least one physical exercise in said database on the basis of said user objective entered by the user, of one or several physical attributes to be reinforced and/or activated as determined according to the user's level for this attribute, and at least one feedback from the user, said feedback indicating the sensation of the user during the performance of a physical exercise.
